(19) United States
(12) Patent Application Publication
(10) Pub. No.: US 2018/0243236 A1
(43) Pub. Date: Aug. 30, 2018

(54) NUTRACEUTICAL COMPOSITION FOR THE TREATMENT OF INFLAMMATION

(71) Applicant: BRANDON HART, DALLAS, TX (US)

(72) Inventor: BRANDON HART, DALLAS, TX (US)

(21) Appl. No.: 15/445,887

(22) Filed: Feb. 28, 2017

(51) Int. Cl. A61K 31/05; A61K 31/015; A61K 9/00; A61K 9/08; A23L 33/00; A23L 33/105; A23L 33/10

(52) U.S. Cl. CPC A61K 31/05; A61K 31/015; A61K 9/0053; A61K 9/0043; A23V 2002/00; A23L 33/30; A23L 33/105; A23L 33/10; A61K 9/08

(57) ABSTRACT

A method treating inflammation is described. The method includes using cannabidiol in an ingestible solution to naturally activate the body's endocannabinoid system and encourage activation of the cannabinoid receptors. The method may include formulating the ingestible solution, extracting cannabidiol in its natural form from a plant, and ingesting the solution into the body. The solution may include other additives that are simply mixed in with the cannabidiol. No adjustments to the natural molecular structure of the ingredients occurs.

### BACKGROUND

### 1. Field of the Invention

The present application concerns pharmaceutical compounds and compositions that are useful in the treatment of inflammation, and more particularly to a nutraceutical composition comprising of phytochemicals for the treatment of pain and discomfort caused by inflammation.

### 2. Description of Related Art

A cannabinoid is one of a class of diverse chemical compounds that acts on cannabinoid receptors in cells that alter neurotransmitter release in the brain. Cannabinoid receptors are located throughout the body. These receptors are part of the Endocannabinoid system which is involved in a variety of physiological processes including appetite, pain-sensation, mood, and memory. By acting upon cannabinoid receptors in the appropriate way, the body's reaction to the physiological processes can be manipulated.

Cannabinoid receptors are of a class of cell membrane receptors under the G protein-coupled receptor superfamily. Cannabinoid receptors are activated by three major groups of ligands: endocannabinoids, produced by the body; phytocannabinoids (plant cannabinoids); and synthetic cannabinoids (such as HU-210) that are manufactured artificially. All of the endocannabinoids and plant cannabinoids are lipophilic, such as fat soluble compounds.

Synthetic cannabinoids encompass a variety of distinct chemical classes: the classical cannabinoids structurally related to THC, the nonclassical cannabinoids (cannabimimetics) including the aminoalkylindoles, 1,5-diarylpyrazoles, quinolines, and arylsulfonamides, as well as eicosanoids related to endocannabinoids.

Current anti-inflammatory products are typically derived from synthetic chemicals and operate to reduce inflammation by inhibiting enzyme activity. In particular, the approach has been to inhibit the COX-1 and COX-2 enzymes in creating inflammation. Although effective, this method has many side effects which can include stomach pain, heartburn, stomach ulcers, increased bleeding, headaches, dizziness, ringing in the ears, liver and kidney problems. Due to these complications, it is not recommended that they be consumed for long periods of time. This creates a challenge for patients as they must now find an alternative method to reduce the suffering from their chronic pain.

Although strides have been made to reduce the inflammation of the body for chronic pain sufferers, considerable shortcomings prevail. A new treatment method is needed that directly/indirectly activates the endocannabinoid system via its receptors by the introduction of a phytochemical/exogenous cannabinoid. The activation of the endocannabinoid system triggers a natural chemical reaction that down regulates the inflammatory process.

### DESCRIPTION OF THE DRAWINGS

The novel features believed characteristic of the application are set forth in the appended claims. However, the application itself, as well as a preferred mode of use, and further objectives and advantages thereof, will best be understood by reference to the following detailed description when read in conjunction with the accompanying drawings, wherein:

FIG. 1 is a chart of the method of treating inflammation according to an embodiment of the present application.

FIG. 2 is chart of an ingredient within an ingestible solution used in the method of treating inflammation of FIG. 1.

FIGS. 3-4 are charts of other forms of the ingestible solution of FIG. 2.

FIG. 5 is a chart showing additional supplemental additives for use in the solutions of FIGS. 2-4.

While the method of the present application is susceptible to various modifications and alternative forms, specific embodiments thereof have been shown by way of example in the drawings and are herein described in detail. It should be understood, however, that the description herein of specific embodiments is not intended to limit the application to the particular embodiment disclosed, but on the contrary, the intention is to cover all modifications, equivalents, and alternatives falling within the spirit and scope of the process of the present application as defined by the appended claims.

### SUMMARY OF THE INVENTION

It is an object of the present application to provide a method of relieving pain and discomfort by reducing inflammation and other pain causing chemicals without the use of synthetic cannabinoids.

It is an object of the present application to provide a new treatment method that activates the endocannabinoid system directly/indirectly to provide a phytochemical that activates receptors through a natural chemical reaction to down regulate the inflammation process.

The method of treating inflammation can include ingesting a pure and natural form of cannabidiol into the body so as to relieve pain and discomfort by reducing inflammation and other pain causing chemicals through natural stimulation methods of the body, the cannabidiol forming an ingestible solution. Cannabidiol may be the only ingredient of the ingestible solution.

Additional ingredients may be mixed in with cannabidiol to assist the body in reducing inflammation. These ingredients may include, but are not limited to, Beta-caryophyllene, Humulene, Myrcene, and Limonene. The ingredients are mixed and not chemically altered or combined.

It is an object of the present application that the manner of consumption may be done orally or through the nasal cavity. Both liquid and vapor forms of the ingestible solution are contemplated. Other forms of administration to a user are topical, intravenously, orally via pill or drink, snorting, or even being infused with an energy drink or other blended drink.

The method of treating inflammation may include extracting cannabidiol in a pure and natural form and formulating an ingestible solution. The ingestible solution includes cannabidiol. Followed by ingesting the ingestible solution into the body so as to relieve pain and discomfort by reducing inflammation and other pain causing chemicals through natural stimulation methods of the body.

### DETAILED DESCRIPTION OF THE PREFERRED EMBODIMENT

Illustrative embodiments of the preferred embodiment are described below. In the interest of clarity, not all features of an actual implementation are described in this specification. It will of course be appreciated that in the development of any such actual embodiment, numerous implementation-specific decisions must be made to achieve the developer's specific goals, such as compliance with system-related and business-related constraints, which will vary from one implementation to another. Moreover, it will be appreciated that such a development effort might be complex and time-consuming but would nevertheless be a routine undertaking for those of ordinary skill in the art having the benefit of this disclosure.

The device and method in accordance with the present application overcomes one or more of the above-discussed problems commonly associated with traditional treatment methods. In particular, the method provides a way of relieving pain and discomfort by reducing inflammation and other pain causing chemicals through natural stimulation methods of the body. Furthermore, the method includes the use of cannabidiol, beta-caryophyllene (BCP) and/or humulene (alpha-caryophyllene) within the body for use as an anti-inflammatory. These activate the endocannabinoid system directly/indirectly to provide a phytochemical that activates receptors through a natural chemical reaction to down regulate the inflammation process. These and other unique features of the method are discussed below and illustrated in the accompanying drawings.

During the normal transmission of pain signals, ascending node septic signals travel up the spinothalamic pathway to the thalamus to be processed and relayed to cortical and other areas. Signals are also relayed to the periaqueductal gray matter which, plays a key role in descending pain modulation, where cannabinoid receptors can be found (brain, lungs, liver, kidney).

Endogenous cannabinoids are created by the phospholipid bio layer of the postsynaptic membrane. These cannabinoids are then released directly into the synaptic cleft where they act in reverse manner on the presynaptic neuron where the cannabinoid receptors are expressed.

When these endogenous cannabinoids bind to the receptor they effect the intracellular signal transduction pathway which lessens the effect of calcium ions into the presynaptic neuron and therefore decreases further release of neurotransmitters. The result is an influence on the frequency of postsynaptic firing.

The introduction of exogenous cannabinoids through oral or sublingual administration provides an increase in cannabinoid receptor activity. Orally, they travel through the digestive tract to the liver where they are metabolized and then enter the bloodstream to begin their journey to the central nervous system. The onset of action from oral administration is less rapid compared to the sublingual route. When exogenous cannabinoids are delivered sublingually they travel through the mucus layer to the mucous membrane where they diffuse rapidly into the circulatory system. They then travel through the blood stream toward the central nervous system where they disseminate across the blood brain barrier and into the surrounding tissues.

Exogenous cannabinoids act upon the same cannabinoid receptors throughout the central and peripheral nervous system as the endogenous cannabinoids.

Exogenous cannabinoids bind to cannabinoid receptors and imitate the endogenous synaptic modulatory effect by decreasing the inflow of calcium ions and down regulating the natural pain process.

Found in all mammals, the endocannabinoid system is made up of millions of cannabinoid receptor sites located primarily throughout the brain and central nervous system and the immune system that act in neural communication. There are currently two known subtypes of cannabinoid receptors, termed CB1 and CB2. The CB1 receptor is expressed mainly in the brain, but also in the lungs, liver and kidneys. The CB2 receptor is expressed mainly in the immune system and in hematopoietic cells. By acting upon and manipulating these receptors in a natural manner, one can affect how the body reacts to appetite, pain-sensation, mood, and memory. Additionally, this is done without the use of synthetic cannabinoids that lead to significant side-effects. The present application discloses a method of treatment for inflammation through natural stimulation of the human body.

The human body is capable of making its own cannabinoids. By introducing a particular plant based cannabinoid into the body that is used to assist and encourage activation of receptors, the body itself works to naturally down regulate inflammation. Anandamide (AEA) and 2-Arachidonoylglycerol (2-AG) are the body's own endocannabinoids, created to control communication between cells, mediating the body's functions. The endocannabinoid system is one of the most important regulatory systems in the human body.

The invention of the present application is composed of essential oils that are rich in specific phytochemicals derived from botanical sources. These phytochemicals target specific receptors in the body that are part of the Endocannabinoid system. By activating these receptors, the body begins a natural process of down regulating specific chemicals that are used to create inflammation and other pain signaling chemicals. The result is a rapid reduction in pain and discomfort without the side effects that are often experienced from traditional nonsteroidal anti-inflammatories.

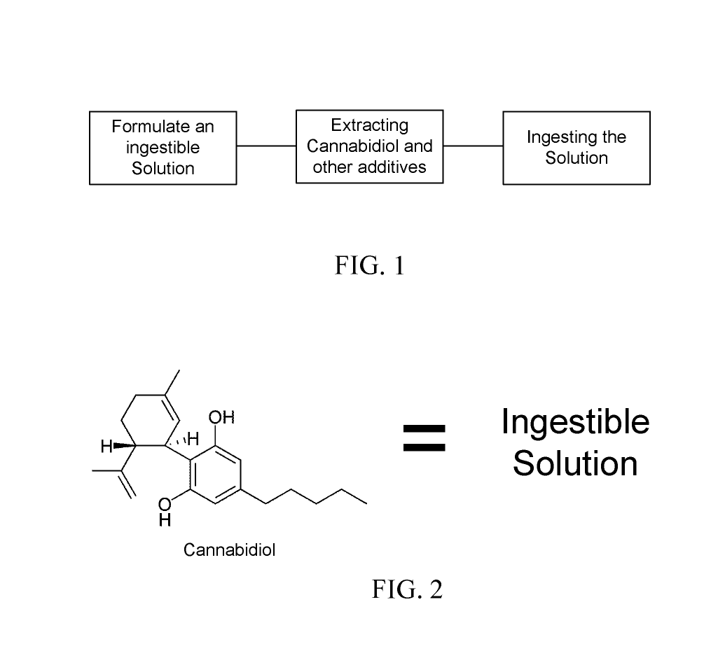

Referring now to the Figures. FIG. 1 illustrates the method of treating inflammation. The method of the present application includes the steps of: 1) extracting cannabidiol and potentially other additives; 2) formulating an ingestible solution; and 3) ingesting the solution into the body. Cannabidiol is the main ingredient and is optionally the only ingredient (see FIG. 2). Ingesting the solution (i.e. cannabidiol) into the body may be done in many different forms. For example, it is preferred that the cannabidiol is consumed in a liquid form, however, other forms are considered feasible, such as vapor. Ingestion broadly refers to the taking of a substance into the body by oral means or through absorption. Cannabidiol may be ingested through sublingual means (i.e. under the tongue), via swallowing, or through the nasal passage of the body. Other known methods may include topical, intravenously, orally via pill or drink, snorting, or even being infused with an energy drink or other blended drink.

A first step of the process includes the need to extract cannabidiol from the plant and to formulate the solution for ingesting. It is desired that the cannabidiol is produced with pure plant oils without synthetic modifications, preservatives, or emulsifiers. Once extracted, the cannabidiol may be ingested in its pure form (the solution being wholly cannabidiol) or may be combined with other natural ingredients. This is seen in FIG. 2 where cannabidiol is shown in its molecular form as the sole ingredient in the ingestible solution. It is important to note that cannabidiol in its pure and natural form may form the basis of the entire solution to be ingested. No other fillers, carriers or additives are necessary.

The ingredient(s) forming the ingestible solution are kept in their pure and natural form. When combined together, the selected proportions of each ingredient are simply mixed together and are not configured to alter or combine so as to form any altered compositions as seen with synthetic cannabinoids. The solution of the present application is done wherein the natural makeup of cannabidiol and other ingredients are unchanged.

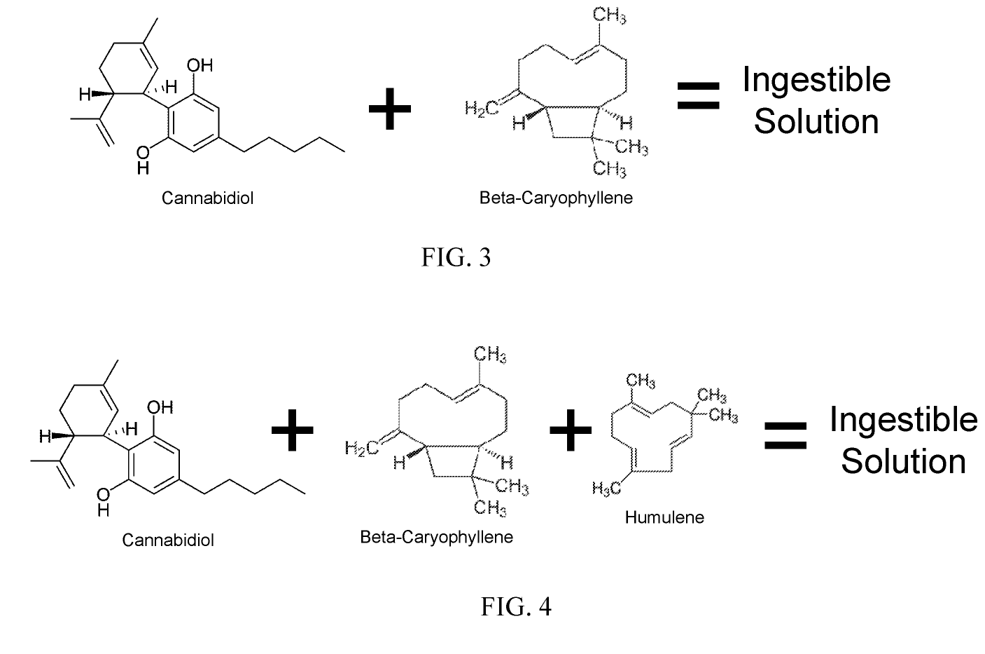

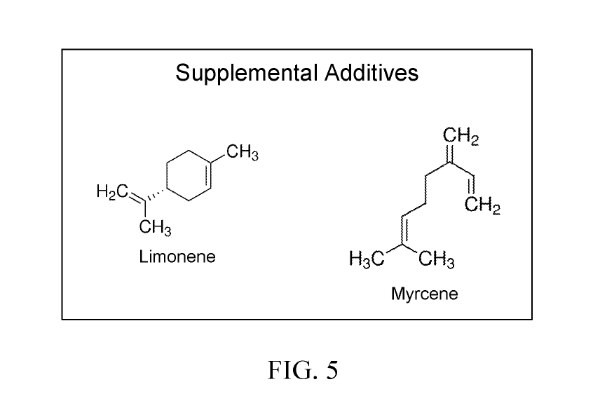

As seen in FIGS. 3-5 other ingredients in their molecular form are shown that may be mixed in with cannabidiol to form an ingestible solution to further assist with inhibiting inflammation. In FIG. 3, beta-caryophyllene is mixed in with the cannabidiol to form the ingestible solution. In FIG. 4, humulene is also added. As a group, these ingredients act to bind to CB2 receptors. The precise proportions of each ingredient are customizable depending on the use and other conditions. FIG. 5 also shows other potential additives, such as Limonene and Myrcene. Although natural additives may be included with cannabidiol, it is understood that cannabidiol is the predominant ingredient in the solution.

The current application has many advantages over the prior art including at least the following: (1) naturally activating the body's own resources to down regulate inflammation; (2) relieve pain naturally; and (3) various methods of ingesting.

The particular embodiments disclosed above are illustrative only and are not intended to be exhaustive or to limit the invention to the precise form disclosed, as the embodiments may be modified and practiced in different but equivalent manners apparent to those skilled in the art having the benefit of the teachings herein. It is therefore evident that the particular embodiments disclosed above may be altered or modified, and all such variations are considered within the scope and spirit of the application. Accordingly, the protection sought herein is as set forth in the description. It is apparent that an application with significant advantages has been described and illustrated. Although the present application is shown in a limited number of forms, it is not limited to just these forms, but is amenable to various changes and modifications without departing from the spirit thereof.

1. A method of treating inflammation, comprising:
ingesting a pure and natural form of beta-caryophyllene into the body so as to relieve pain and discomfort by reducing inflammation and other pain causing chemicals through natural stimulation methods of the body, the beta-caryophyllene forming an ingestible solution.

2. (canceled)

3. The method of claim 1, further comprising:
mixing into the ingestible solution a proportion of humulene.

4. The method of claim 1, further comprising:
mixing into the ingestible solution an additive.

5. The method of claim 1, further comprising:
ingesting the solution may occur through oral consumption.

6. The method of claim 1, further comprising:
ingesting the solution may occur through nasal consumption.

7. A method of treating inflammation, comprising:
extracting beta-caryophyllene in a pure and natural form;
formulating an ingestible solution having predominantly beta-caryophyllene, the ingestible solution devoid of cannabidiol; and
ingesting the ingestible solution into the body so as to relieve pain and discomfort by reducing inflammation and other pain causing chemicals through natural stimulation methods of the body.

8. (canceled)

9. (canceled)

10. The method of claim 1, wherein the ingestible solution further includes humulene.

11. The method of claim 1, wherein the ingestible solution includes a natural additive.
